(19) United States
(12) Patent Application Publication
(10) Pub. No.: US 2005/0249666 A1
(43) Pub. Date: Nov. 10, 2005

(54) Establishment of human cancer cell lines with metastatic potential using NOD/SCID

(75) Inventors: Masato Nakamura, Tokyo (JP); Yasuyuki Ohnishi, Yokohama (JP); Hiroshi Suemizu, Isehara (JP); Makoto Monnai, Yokohama (JP)

Correspondence Address: HELLER EHRMAN LLP, 275 MIDDLEFIELD ROAD, MENLO PARK, CA, 94025-3506, US

(73) Assignees: CENTRAL INSTITUTE FOR EXPERIMENTAL ANIMALS, Kawasaki-shi (JP); CENTER FOR THE ADVANCEMENT OF HEALTH AND BIOSCIENCE, USA, Menlo Park, CA (US)

(21) Appl. No.: 10/955,192

(22) Filed: Sep. 29, 2004

Related U.S. Application Data
(63) Continuation-in-part of application No. 10/871,186, filed on Jun. 18, 2004.
(60) Provisional application No. 60/487,044, filed on Jul. 10, 2003.

(51) Int. Cl. A61K 49/00

(52) U.S. Cl. 424/9.2

(57) ABSTRACT

The invention provides a new reproducible transgenic mouse model for the study of tumor metastasis. In particular, the invention concerns the study of tumor metastasis in a NOD/SCID/γ_{c}^{null} transgenic mouse model.

This application is a continuation-in-part of copending U.S. application Ser. No. 10/871,186 filed on Jun. 18, 2004, which claims the benefit of the priority under 35 U.S.C. § 119(e) of provisional application Ser. No. 60/487,044 filed on Jul. 10, 2003.

### BACKGROUND OF THE INVENTION

1. Field of the Invention

The present invention concerns a transgenic animal model for the analysis of tumor metastasis. In particular, the present invention provides methods for the study of tumor metastasis, including the analysis of metastasis of cancer, in a transgenic (including knock out) mouse model. In addition, the present invention concerns the establishment of human cancer cell lines with high metastatic potential in NOD/SCID/γ_{c}^{null} (NOG) mice.

2. Related Art

Immunodeficient mice, such as athymic nude mice, C.B-17/severe combined immunodeficiency (scid) mice and NOD/SCID mice have been widely used as animal models in cancer metastasis research (Bruns et al., Int. J. Cancer 10:102(2):101-8 (2002); Ohta et al., Jpn. J. Cancer Chemother. 23:1669-72 (1996); Jimenez et al., Ann. Surg. 231:644-54 (2000)). Thus, such mouse models have been used for preclinical testing of new cancer drugs and for the detection of metastasis related genes (Bruns et al., supra; Ohta et al., supra; Jimenez et al. supra; Hotz et al., Pancreas 26:E89-98 (2003); Tarbe et al., Anticancer Res. 21:3221-8 (2001)). However, the use of these models for studying the metastases of human cancer cells has so far been limited, primarily due to the low efficiency of the incidence of cancer metastasis in the recipient mice, and the large cell number required to achieve the desired results.

Recently, to establish a more efficient animal recipient for xenotransplantation, a novel immunodeficiency mouse, NOD/SCID/γ_{c}^{null} (also referred to as NOD/ShiJic-scid with γ_{c}^{null}, or NOG) has been developed. NOG transgenic mice have been described as an excellent recipient mouse model for engraftment of human cells (Ito et al., Blood 100:3175-82 (2002)), and for the study of the in vivo development of human T cells from CD34(+) cells (Saito et al., Int. Immunol. 14:1113-24 (2002)). When human cord blood stem cells (CBSC) were preserved in NOG mice, CBSC were differentiated to T lymphocytes and migrated to the peripheral lymphoid organs (Yahata et al., J. Immunol. 169:204-9 (2002)).

Metastasis, including hepatic metastasis, is often observed in human cancer, including pancreatic cancer even in early stage, cancers of the digestive tract, including colorectal cancer and gastrointestinal cancer, lung cancer, and the like, and is one of the most frequent causes of cancer deaths. New strategies are necessary to manage cancer metastases, which, in turn, require the availability of appropriate and efficient animal models, and cell lines with high metastatic potential for study of tumor metastasis and for testing drug candidates for the treatment of tumor metastasis, including metastasis in the liver.

### SUMMARY OF THE INVENTION

In one aspect, the present invention concerns a method for testing tumor metastasis, comprising the steps of
(a) inoculating a tumor cell from a metastatic tumor or tumor cell line into a NOD/SCID/γ_{c}^{null} mouse, and
(b) monitoring the development of tumor metastasis.

In one embodiment, the tumor is cancer, such as, for example, pancreatic cancer, prostate cancer, breast cancer, colorectal cancer, gastrointestinal cancer, colon cancer, lung cancer, hepatocellular cancer, cervical cancer, ovarian cancer, liver cancer, bladder cancer, cancer of the urinary tract, thyroid cancer, renal cancer, carcinoma, melanoma, or brain cancer.

In another embodiment, the metastasis is hepatic, bone, brain or lung metastasis, in particular, hepatic metastasis.

In yet another embodiment, the tumor cell is from a metastatic tumor cell line, which can, for example, be a strongly, moderately or lightly metastatic tumor cell line.

Pancreatic cancer cell lines suitable for the present invention include, for example, MIAPaCa-2, AsPC-1, PANC-1, Capan-1, and BxPC-3.

Inoculation can be performed, for example, by portal vein injection.

In one embodiment, at least about 1×10² cells are inoculated, without any other pretreatment including irradiation or cytokine-medication.

In another embodiment, at least about 1×10³ cells are inoculated.

In yet another embodiment, at least about 1×10⁴ cells are inoculated.

The development of tumor metastasis can be monitored by methods known in the art, such as by observing the appearance and number of the metastatic nodules formed.

In another aspect, the invention concerns a method for testing a candidate anti-metastasis compound, comprising
(a) administering said candidate compound to a NOD/SCID/γ_{c}^{null} mouse which has developed tumor metastasis, and
(b) monitoring the effect of said candidate compound on said tumor metastasis.

The test compound can be any kind of molecule, including, without limitation, a peptide, polypeptide, antibody or a non-peptide small molecule.

In a further aspect, the invention concerns a method comprising:
(a) introducing into a NOD/SCID/γ_{c}^{null} mouse a foreign gene, and
(b) monitoring the expression of the foreign gene in the mouse.

The foreign gene can be introduced into the mouse by any method of gene transfer, including, without limitation, by a viral vector.

In a particular embodiment, the foreign gene is a gene which is differentially expressed in tumor metastasis, such as hepatic metastasis.

The gene can, for example, be selected from TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); small inducible cytokine subfamily A member 1 (SCYA1). transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene.

In a particular embodiment, the mouse carrying a gene marker of tumor metastasis is treated with a candidate anti-metastasis compound, and the expression level of the gene marker or its expression product as a result of the treatment is monitored.

The invention further concerns a cell line of human pancreatic cancer cells, having the metastatic and gene expression characteristics of BxPC-3LM1.

In a different aspect, the invention concerns a method of screening a potential therapeutic agent for the treatment of cancer, including, but not limited to metastatic cancer, such as metastatic pancreatic cancer, comprising administering the potential therapeutic agent to a cell line having the metastatic and gene expression characteristics of BxPC-3LM1, culturing the cells of the cell line, and determining whether the potential therapeutic agent inhibits the growth of the cells, proliferation of the cells or tendency of the cells to metastasize.

In a particular embodiment, the cell line is BxPC-3LM1.

In yet another aspect, the invention concerns a method of screening potential therapeutic agents for the treatment of cancer, including, but not limited to metastatic cancer, such as metastatic pancreatic cancer, in vivo comprising administering cells of a cell line having the metastatic and gene expression characteristics of BxPC-3LM1 to a mammalian host, allowing the cells to proliferate in the host, and administering the therapeutic agent to the host, and examining the host to determine whether the therapeutic agent inhibits the growth, proliferation or metastasizing of the pancreatic cancer cells.

Again, in a particular embodiment, the cell line is BxPC-3LM1.

In another embodiment, the mammalian host is a mouse, such as a NOG mouse.

### BRIEF DESCRIPTION OF THE DRAWINGS

FIGS. 1A and B illustrate the incidences of hepatic metastasis and the number of liver foci in NOG mice following the inoculation of 1×10⁴, 1×10³ and 1×10² cells of the indicated pancreatic adenocarcinoma cells lines (MIAPaCa-2, AsPC-1, PANC-1, Capan-1, and BxPC-3.

FIG. 2. Establishment of a the highly metastatic cell line, BxPC-3LM1, derived from the low metastatic cell line, BxPC-3.

FIG. 3. PCR data confirming that BxPC-3LM1 is a cell line of human origin, using β-actin specific primers.

FIG. 4. Microscopic view of the cell lines BxPC-3 and BxPC-3LM1 in vitro.

FIG. 5. Overview of establishing and testing the cell line BxPC-3LM1.

FIG. 6 is a scatter graph of microarray analysis of the BxPC-3LM1 and BxPC-3 cell lines. Signal intensity of each spot of gene chip microarray (gene expression) was plotted. The genes in the blue bottom area was omitted from further analysis due to low intensity. The genes in the red are were subjected to further analysis.

FIG. 7. To confirm the results of gene expression analysis, some genes which showed significant difference in their expression levels in BxPC-3 and BxPC-3LM1 were subjected to amplification by RT-PCR. The Figure shows the results of the RT-PCR analysis after varying numbers of amplification cycles.

Table 1. Hepatic metastasis after intrasplenic injection of various human pancreatic adenocarcinoma cell lines.

Table 2. Genes differentially expressed in cell lines with high metastasis potential relative to cell lines with low metastatic potential.

Table 3. Metastatic potentials of BxPC-3 and BxPC-3LM1 in the liver of NOG mouse.

Table 4 Metastatic potentials of BxPC-3 and BxPC-3LM1 in the liver of NOD/SCID mouse.

Table 5 Comparison of characteristics of BxPC-3 and BxPC-3LM1 cell lines.

Table 6 List and signal log ratio of genes differentially expressed between the BxPC-3 and BxPC-3LM1 cell lines, selected by microarray analysis.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma (epithelial), such as, pancreatic cancer, prostate cancer, breast cancer, colorectal cancer, gastrointestinal cancer, colon cancer, lung cancer, hepatocellular cancer, cervical cancer, ovarian cancer, liver cancer, bladder cancer, cancer of the urinary tract, thyroid cancer, renal cancer, melanoma, and brain cancer; and sarcoma (non-epithelial), such as, liposarcomas, leiomyosarcomas, rhabdomyosarcoma, synovial sarcoma, angiosarcoma, fibrosarcoma, malignant peripheral nerve tumor, gastrointestinal stromal tumor, desmoid tumor, Ewing's sarcoma, osteosarcoma, chondrosarcoma, leukemia, lymphoma and myeloma.

The term “metastasis” is used herein in the broadest sense and refers to the spread of tumor, e.g. cancer from one part of the body to another. Tumors formed from cells that have spread are called secondary tumors, and contain the same type of cells as the original (primary) tumor. Thus prostate cancer that has metastasized to liver or bone is not liver or bone cancer, rather metastasized prostate cancer, as it still contains prostate cancer cells, regardless of their location.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The terms “differentially expressed gene,” “differential gene expression” and their synonyms, which are used interchangeably, refer to a gene whose expression is at a higher or lower level in one cell or cell type relative to another, or one patient or test subject relative to another. Thus, for example, differential gene expression can occur in normal cell/tissue/patient relative to a corresponding diseased cell/tissue/patient, or can reflect differences is gene expression pattern between different cell types or cells in different stages of development. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may, for example, be evidenced by a change in mRNA levels, surface expression, or secretion or other partitioning of a polypeptide. Differential gene expression may include a comparison of expression between two or more genes or their gene products, or a comparison of the ratios of the expression between two or more genes or their gene products, or a comparison of two differently processed products of the same gene. For the purpose of the present invention, “differential gene expression” is considered to be present when there is at least an about 2-fold, preferably at least about 2.5-fold, more preferably at least about 4-fold, even more preferably at least about 6-fold, most preferably at least about 10-fold difference between the expression of a given gene or gene product between the samples compared.

The term “microarray” refers to an ordered arrangement of hybridizable array elements on a substrate. The term specifically includes polynucleotide microarrays, such as cDNA and oligonucleotide microarrays, and protein arrays. In a particular embodiment, a microarray is an array of thousands of individual gene (DNA) sequences immobilized in a known order on a solid support. RNAs from different tissues are hybridized to the DNA on the chips. An RNA molecule will only bind to the DNA from which it was expressed. As a result, the relative expression of thousands of genes in biological samples (e.g. normal and diseased tissue, tissue treated or untreated with a certain drug, etc.) can be compared in a single assay. In a similar protein sequences can be displayed on a microarray chip and used to study protein-protein interactions, or differences in protein levels in different biological samples, e.g. tissues.

The term “polynucleotide,” generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein includes triple-stranded regions comprising RNA or DNA or both RNA and DNA. The strands in such regions may be from the same molecule or from different molecules. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs.

The terms “transgenic animal” and “transgenic mouse” as well we their grammatical equivalents, are used to refer to animals/mice deliberately produced to carry a gene from another animal.

The term “xenotransplantation” is used in the broadest sense and refers to the transfer of living cells, tissues or organs from one animal species into another, including humans.

B. Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “Transgenic Mouse: Methods and Protocols” (Methods in Molecular Biology, Clifton N.J., Vol. 209, M. H. Hofker et al., eds.).

The present invention provides a sensitive and reliable transgenic animal model for the study of tumor metastasis. In particular, the present invention provides a reproducible mouse model of hepatic metastasis, which involves the introduction of mammalian (e.g. human) cancer cells into NOG mice.

NOG mice were developed at the Central Institute for Experimental Animals (CIEA, Kawasaki, Japan), and are also described in co-pending U.S. application Ser. No. 10/221,549 filed on Oct. 25, 2001, and in PCT Publication No. WO 03/0182671, the entire disclosures of which are hereby expressly incorporated by reference.

In brief, to establish an improved animal recipient for xenotransplantation, NOD/SCID/γ_{c}^{null} (NOG) mice double homozygous for the severe combined immunodeficiency (SCID) mutation and interleukin-2Rγ (IL-2Rγ) allelic mutation (γ_{c}^{null}) were generated by 8 backcross matings of C57BL/6J-γ_{c}^{null} mice and NOD/Shi-scid mice. When human CD34+ cells from umbilical cord blood were transplanted into this strain, the engraftment rate in the peripheral circulation, spleen, and bone marrow were significantly higher than that in NOD/Shi-scid mice treated with anti-asialo GM1 antibody or in the β2-microglobulin-deficient. NOD/LtSz-scid (NOD/SCID/β₂ₘ^{null}) mice, which were as completely defective in NK cell activity as NOD/SCID/β₂ₘ^{null}) mice. The same high engraftment rate of human mature cells was observed in ascites when peripheral blood mononuclear cells were intraperitoneally transferred. In addition to the high engraftment rate, multilineage cell differentiation was also observed. Further, even 1×10(2) CD34+ cells could grow and differentiate in this strain. Based on these results, the NOD/SCID/γ_{c}^{null} mice were described to be superior animal recipients for xenotransplantation, especially for human stem cell assays. For further details see, e.g. Hiramatsu et al., Blood 100:3175-82 (2002).

It has now been found that the NOG mice are a superior mouse model for the study of human cancer metastasis. As such, this model can be used, for example, to screen and evaluate anti-cancer drugs and anti-metastasis drug candidates, and for the detection/screening of genes related to cancer metastasis, which, in turn, find utility in the diagnosis and/or treatment of metastatic cancer, and related conditions, including gene therapy treatment of metastatic cancer.

The mouse model of the present invention is suitable for modeling and studying any kind of metastasis, including hepatic, bone, brain, and lung metastasis. Metastasis occurs in all types of cancers, including, without limitation, pancreatic cancer, prostate cancer, breast cancer, colorectal cancer, gastrointestinal cancer, colon cancer, lung cancer, hepatocellular cancer, cervical cancer, ovarian cancer, liver cancer, bladder cancer, cancer of the urinary tract, thyroid cancer, renal cancer, carcinoma, melanoma, and brain cancer. Although the invention will be illustrated by analyzing hepatic metastasis of human pancreatic cancer, it is not so limited. The NOG mouse model can also be used to study metastases originating from other types of cancer at any location, including liver, bone, brain and liver.

Methods of xenotransplantation are well known in the art, and described, for example, in the following references, which are incorporated herein in their entirety: Fiebig et al., “Human Tumor Xenografts: Predictivity, Characterization and Discovery of New Anticancer Agents,” in Contributions to Oncology: Relevance of Tumor Models for Anticancer Drug Development, Fiebig & Burger, eds. (Basel, Karger 1999), vol. 54, pp. 29-50; Berger et al., “Establishment and Characterization of Human Tumor Xenografts in Thymus-Aplastic Nude Mice,” in Immunodeficient Mice in Oncology, Fiebig & Berger, eds. (Basel, Karger 1992), pp. 23-46; Fiebig & Burger, “Human Tumor Xenografts and Explants,” in Models in Cancer Research, Teicher, ed. (Humana Press 2002) pp. 113-137.

A specific method of xenotransplantion is also described in Example 1 below.

In general, mammalian tumor specimens, preferably human tumor specimens, may be obtained and implanted into mice, preferably athymic nude mice. The tumor specimens may be obtained by any method known in the art. In one embodiment the tumor specimens are surgically resected, such as in a biopsy or in the process of surgery to remove the tumor from the mammal. In another embodiment the tumor specimen is obtained by purifying circulating tumor cells from the mammals blood.

Typically, cancer cells are transplanted into mice via tail vein injection, with or without prior immune-suppression, such as a sublethal dose of whole body irradiation and/or the administration of an immunosuppressant. For study of hepatic metastases, the cancer cells may be introduced into the animals by intrasplenic (portal vein) injection using an appropriate indwelling catheter. Pulmonary metastasis can be established, for example, by intravenous injection of tumor cells into the recipient animals, for example as described in Worth and Kleinerman; Clin Exp. Metastasis 17:501-6 (1999). The tumor cells may originate from tumor (cancer) cell lines, and from primary tumors (e.g. cancer) obtained from human or non-human subjects.

To study bone metastasis, macroscopic fragments of human fetal bone or mouse bone, may be implanted into NOG mice. A few weeks later, human tumor (cancer) cell lines or cells of primary tumors (cancer) can be injected either intravenously (colonization assay), or directly into the implanted tissue fragments. Tumor metastasis can be monitored by methods known in the art, including various imaging techniques and histologic examination.

When used for drug screening, following the engraftment of xenogenic tumor cells (either from cell lines or from primary tumors), the NOG mice that have developed metastatic cancer can be treated with the test compound(s), and any change in the number, size or other properties of the metastatic nodules as a result of drug treatment, and the viability of the test animals are monitored relative to untreated and/or positive control, where the positive control typically is an animal treated with a know anti-metastatic compound. The administration of the test compounds can be performed by any suitable route, including, for example, oral, transdermal, intravenous, infusion, intramuscular, etc. administration. Results obtained in this model can then be validated by follow-up pharmacokinetic, toxicologic, biochemical and immunologic studies, and ultimately human clinical studies.

The NOG mouse model can also be used to study targeted gene delivery to metastatic nodules in vivo, for example by portal vein infusion of a retroviral vector. In particular, this NOG model can be used to study the feasibility of gene transfer to target tumor metastasis, to monitor the duration and level of gene expression and the degree of therapeutic effect, to optimize the dosing regimen and/or mode of administration, to study the dissemination of the gene transfer vector to non-targeted tissues (which provides information about potential toxicity), and the like.

Gene delivery most commonly is performed using retroviral vectors by techniques well known in the art. Retroviruses are enveloped viruses containing a single stranded RNA molecule as their genome. Following infection, the viral genome is reverse transcribed into double stranded DNA, which integrates into the host genome where it is expressed. The viral genome contains at least three genes: gag (coding for core proteins), pol (coding for reverse transcriptase) and env (coding for the viral envelope protein). At each end of the genome are long terminal repeats (LTRs) which include promoter/enhancer regions and sequences involved with viral integration. In addition there are sequences required for packaging the viral DNA and RNA splice sites in the env gene. Retroviral vectors used in mouse models are most frequently based upon the Moloney murine leukemia virus (Mo-MLV). In addition, lentiviruses can, for example, be used for gene transfer into experimental animals, such as NOG mice.

Gene delivery can also be performed by adenoviral vectors. Adenoviroses are non-enveloped, icosahedral viruses with linear double-stranded DNA genomes. Adenoviruses infect non-dividing cells by interacting with cell surface receptors, and enter cells by endocytosis. Since the genome of adenoviruses cannot integrate with the host cell genome, the expression from adenoviral vectors is transient.

Further details of the invention are illustrated by the following non-limiting examples.

### EXAMPLE 1

Study of Hepatic Metastasis of Human Pancreatic Cancer

Materials and Methods

Male NOG mice and NOD/shiJic-scid mice of 7-9 weeks, which had been obtained from the Central Institute for Experimental Animals (CIEA, Kawasaki, Japan), were used in this study. The animals were kept under specific pathogen-free conditions according to the Guideline for the Regulation of Animal Experimentation of CIEA. All human pancreatic cancer cell lines used in this study were obtained from the American Type Culture Collection (Rockville, Md., USA). Culture media for AsPC-1 and Capan-1 were Dulbecco's modified Eagle's medium (DMEM) supplemented with 20% and 15% fetal bovine serum (FBS, Hyclone), respectively. MIAPaCa-2 and PANC-1 were maintained a culture of DMEM supplemented with 10% FBS. BxPC-3, Capan-2 and PL45 were maintained a culture of RPMI1640 (SIGMA, Cat. No. D6046 or D5796) supplemented with 10% FBS. These were maintained at 37° C. in humidified atmosphere with 5% CO2. Experimental liver metastases were generated by intrasplenic/portal injection of cancer cells, as described previously (Khatib et al., Cancer Res. 62:242-50 (2002)). The animals were sacrificed 6-8 weeks later and liver metastases were enumerated immediately, without prior fixation. The metastatic lesions were evaluated on the following scale: O=No metastatic lesion; 1=1-10 metastatic lesions; 2=11-20 metastatic lesions; 3=21 or more metastatic lesions.

Results

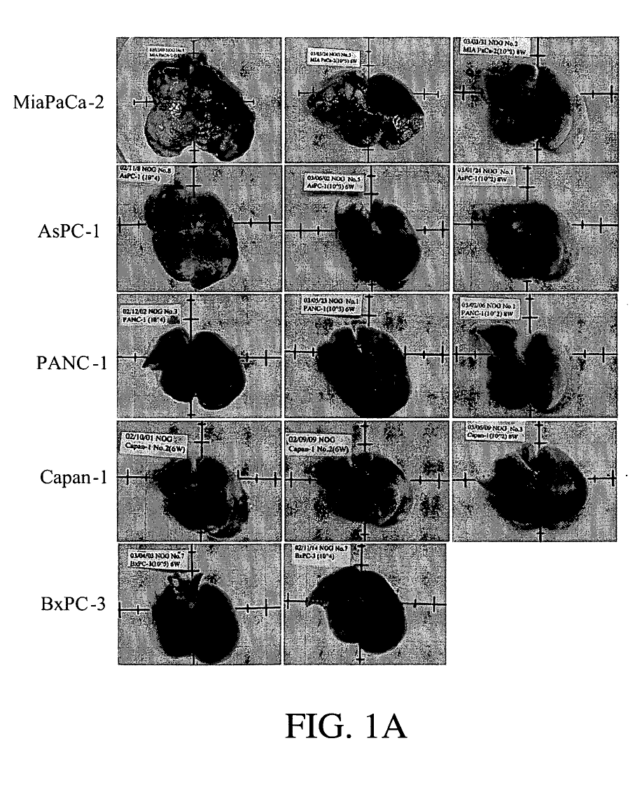

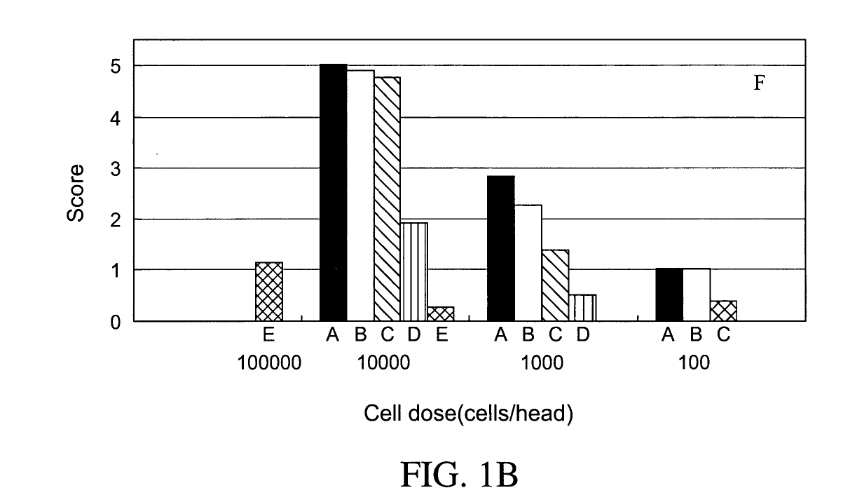

The incidences of hepatic metastases and the number of liver foci in NOG mice were far higher than those in NOD/SCID mice (Table 1 & FIGS. 1A and B). When the mice were inoculated with 1×10⁴ cells and sacrificed 6 weeks later, the incidences of hepatic metastases in NOG mice were as follows:
MIAPaCa-2, AsPC-1 and PANC-1 100%;
Capan-1 90%,
BxPC-3 12.5%; and
PL45 and Capan-2 0%.

In addition, metastases were apparent in 50-80% of NOG mice when 1×10³ MIAPaCa-2, AsPC-1, PANC-1 and Capan-1 cells were inoculated, and even when 1×10² MIAPaCa-2, AsPC-1 and PANC-1 cancer cells were inoculated, 37.5-71.4% of NOG mice show hepatic metastasis. These data indicate that the hepatic metastatic lesions in NOG mice inoculated with human pancreatic cancer cell lines were reproducibly formed in a dose dependent manner.

Typical macroscopic views of liver metastases in NOG mice and in NOD/SCID mice are shown in FIG. 1A. The NOG mice injected with MIAPaCa-2, AsPC-1, PANC-1, Capan-1 and BxPC-3 cells showed multiple round metastases in the liver. However, the numbers of foci in these cell lines were wildly different depending on each cell line. Five out of 7 pancreatic cancer cell lines showed the metastatic potentials in NOG mouse, in contrast, no NOD/SCID mice showed hepatic metastasis under similar conditions, except for AsPC-1. As shown in FIGS. 1A and B, AsPC-1 showed the metastatic potentials in both mice lines, however, the degree of metastases in NOG mice were more severe than those in NOD/SCID mice.

Kusama et al. (Gastroenterology 122:308-17 (2002)) reported that metastatic lesions were apparent in 100% of athymic nude mice injected with 1×10⁶ AsPC-1 cells. These findings suggest AsPC-1 may be one of the cells with high metastatic potential, where the potential is dependent on the cell numbers injected.

The metastatic incidences of NOG mice inoculated with Capan-1 or BxPC-3 were faded away with decreasing the number of inoculating cells. In contrast, metastatic incidences were apparent in more than 50% of NOG mice inoculated with MIAPaCa-2 or AsPC-1 even when NOG mice were inoculated with only 1×10² cells (Table 1). These findings clearly indicate that NOG mice represent a highly superior metastasis model relative to other immunodeficient mouse models, and in particular NOD/SCID mice.

Most previous publications concerning hepatic metastases of human pancreatic cancer cells using nude mice report the intrasplenal inoculation of more than one million cancer cells (Shishido et al., Surg. Today 29(6):519-25 (1999); Nomura et al., Clin. Exp. Metastasis 19:391-9 (2002); and Ikeda et al., Jpn. J. Cancer Res. 81:987-93 (1990)). There are few reports of 100% metastatic incidences, unless high metastatic clones derived from those cells lines were established. However, it is unlikely that more than 1 million cancer cells enter the liver at a stretch via the portal vein and form metastatic foci in pancreatic cancer patients, therefore, the current metastatic animal models are not representative of a typical human clinical situation.

In contrast, NOG mic represent an effective cancer metastasis model, which properly reflects the clinical conditions and behavior of human pancreatic cancer. Accordingly, the well-organized and reproducible hepatic metastases seen in NOG mice are useful in the study of hepatic metastasis of human pancreatic cancer and are expected to become the preferred model for screening and developing new anti-metastasis drugs.

It was reported that the murine NK activity were compensatory very high in immunodeficient animals such as nude, SCID and NOD/SCID mice, and contributed to the low rate of tumor growth and cancer metastasis (Shpitz et al., Anticancer Res. 14(5A):1927-34 (1994)). In contrast, Ito et al. (Blood 100:3221-8 (2001)) reported that NOG mice have no T, B and NK cells and decrease macrophage functions and dendritic cells functions. It is suggested that in the metastasis model using NOG mice, the metastatic potentials of cancer cells are detected without complex effects upon the immune system of the host, especially NK activity.

Conclusions

The data presented demonstrate that the NOD/SCID/γ_{c}^{null} mouse model has a high potential to engraft xenogenic cells. Using this model for intrasplenic (portal vein) injection of cancer cells, reliable hepatic metastasis behavior of human pancreatic cells was observed. Four out of seven cell lines showed high hepatic metastatic potential (>80% incidence), and three of the cell lines studied showed low metastatic potential (<20% incidence) in NOG mice 6 weeks after transplantation only with 1×10⁴ cells. Moreover, hepatic metastases were apparent in NOG mice even when 1×10² cells of high metastatic cell lines were inoculated. Thus, the metastatic ability of cancer cells was demonstrated with a wide range of inoculated cell number, extending through 3 logarithmic orders of magnitude. The results also show that the NOG mouse model is clearly superior over the NOD/SCID model, which is currently considered the optimal animal model for study of cancer metastasis.

### EXAMPLE 2

Detection of Cancer Metastasis Related Genes in cDNA Microarray

Materials and Methods

Human pancreatic tumor cell lines, MIAPaCa-2, Panc1, Capan2 and PL45 (available from ATCC) were cultured according to the method described in Example 1. Total RNA was extracted from confluent culture of those cells using TRIZOL reagent (GIBCO BRL). Cy-3 labeled cDNA probes were synthesized from 20 μG of total RNA using Atlas human 1K specific primer set (BD), PowerScript labeling kit (BD), and Cy-3 fluorochrome (Amersham). Then, the probe was hybridized to the Atlas Glass Human 1.0 Microarray (BD) according to manufacturer's instructions.

The differentially expressed genes among the pancreatic tumor cell lines were globally searched using the Atlas Glass Human 1.0 Microarray (BD). The Cy-3 labeled signals were detected and obtained and analyzed the corresponding images by a GM418 array scanner (Takara). The data processing was carried out using Imagene Version 5.5 software. In this experiment, we classified human pancreatic tumor cell lines into two groups based on their metastatic potential. MIAPaCa-2 and Panc1 cell lines were classified into a highly metastatic group, while the other cell lines, Capan2 and PL45, were classified into a non-metastatic group. To compare the expression profiles, the average of the signal values from the “highly metastatic group” array was divided by the average of the signal values from the “non-metastatic group” array. The resulting values are referred to as “gene expression levels”, where a 10-fold difference and higher values were considered significant.

Results

Gene expression profiles of each cell line were recorded in an EXCEL file (ArrayData.xcl). The genes that were over-expressed in the highly metastatic cell lines (MIAPaCa-2 and Panc1) relative to the non-metastatic cell lines (Capan2 and PL45), and genes that were under-expressed in the highly metastatic cell lines relative to the non-metastatic cell lines are listed in Table 2. For example, butyrate response factor 1 gene (BRF1) was expressed over 100,000 times more in cancer cells in the highly metastatic group than in cells in the non-metastatic group. In contrast, over 100,000 times over-expression of transducing-beta-2 subunit gene was seen in cells of the non-metastatic group.

As shown in Table 2, the following genes are significantly over-expressed in highly metastatic cells relative to non-metastatic cells: TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); and small inducible cytokine subfamily A member 1 (SCYA1).

As shown in Table 2, the following genes are significantly under-expressed in highly metastatic cells relative to non-metastatic cells: transducin P2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene.

The differential expression of the listed and other genes can be used, for example, in drug screening, to test anti-cancer and/or anti-metastatic drug candidates, and for diagnostic and therapeutic purposes, e.g. using gene transfer approaches.

### EXAMPLE 3

Establishment of a Cell Line with High Metastatic Potential

Example 1 describes the establishment of a hepatic metastatic panel using human pancreatic cancer cells xeno-transplanted into NOG mice. Using this panel, the metastatic potentials of several cell lines have been characterized as shown in Table 1. One of the cell lines characterized is BxPC-3. After intrasplenic injection of this cell line into NOG mice only one in eight mice developed hepatic metastasis, i.e. the metastatic potential of this cell line was only 12.5%.

The present Example describes the development of a cell line with high metastatic potential from BxPC-3.

Materials and Methods

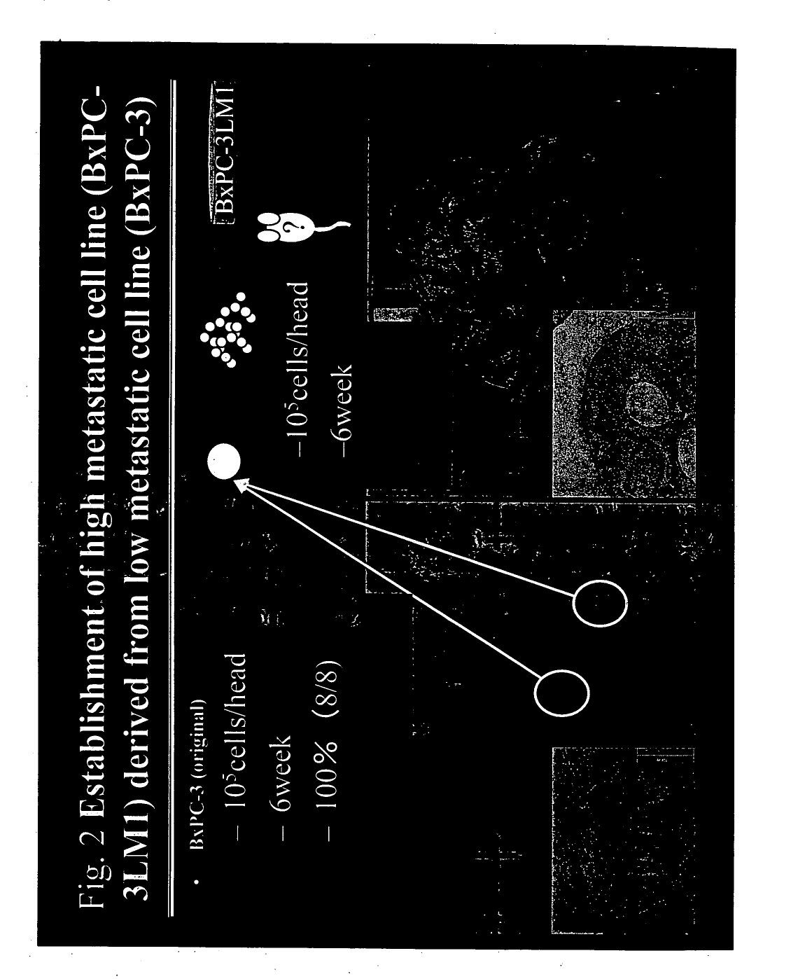

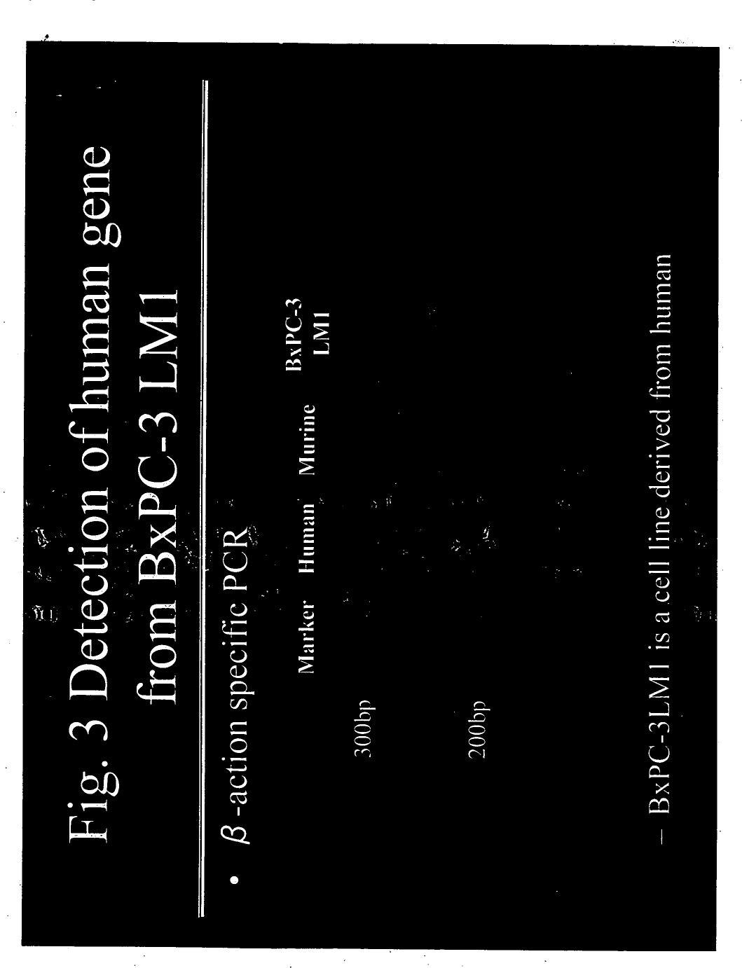

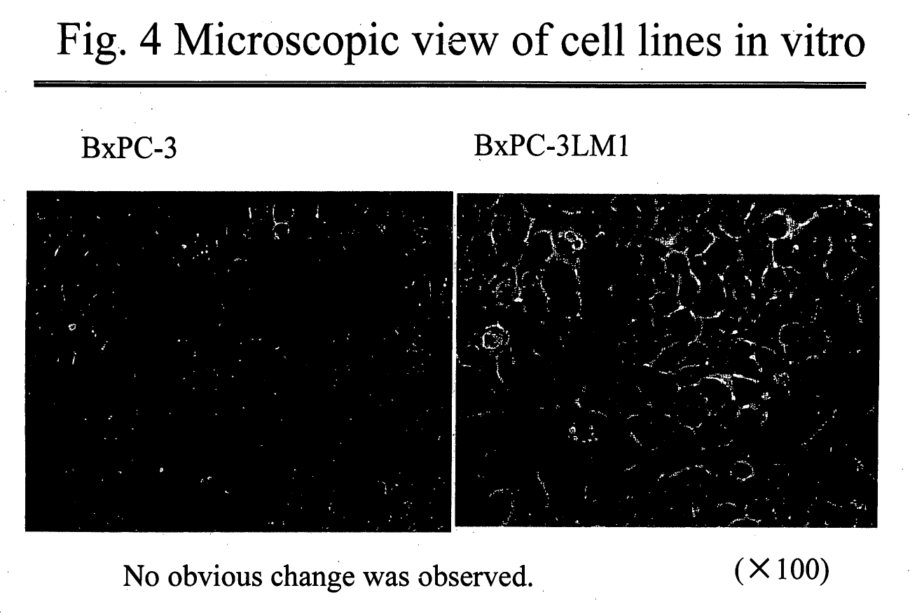

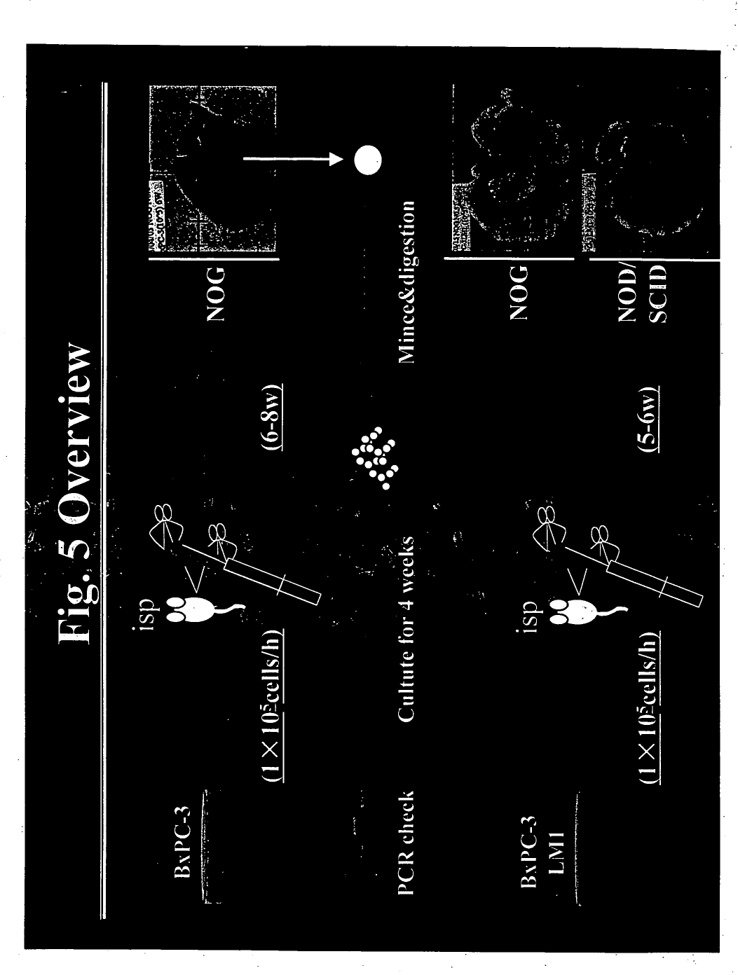

BxPC-3 (1×10⁵ cells) was injected into the spleens of NOG mice. This is barely the amount needed to induce metastasis. After 6-8 weeks, the mice were sacrificed and the livers with a few metastatic foci were harvested. A single cell suspension was prepared by mincing and enzymatic dissociation, and were then cultured in vitro for 4 weeks. The cells in this culture were designated BxPC-3LM1, and the procedure is illustrated in FIGS. 2 and 5.

Results

When the BxPC-3LM1 cell line was analyzed for liver metastatic ability in NOG mice again, high liver metastatic ability was proven (FIG. 2). Table 3 compares the metastatic potentials of the original cell line BxPC-3 and the sub-line BxPC-3LM1 in the liver of the NOG mouse.

In agreement with the data shown in Table 1, 1×10⁴ cells of BxPC-3 resulted in weak metastasis (metastatic score I) in one out of 8 mice, which corresponds to a metastatic incidence of 12.5%. The same dose of BxPC-3ML1 resulted in strong metastasis (scores II and III) in all six mice tested, which translates to a metastatic incidence of 100%.

At a 1×10⁵-cell dose of BxPC-3 resulted in weak liver metastasis in all 8 mice tested (metastatic score: 100%). The same dose of BxPC-3ML1 also yielded a 100% incidence of metastasis, however, with the important difference that all metastases were strong (grade III).

Table 4 compares the metastatic potentials of the original cell line BxPC-3 and the sub-line BxPC-3LM1 in the liver of the NOD/SCID mouse. In this mouse model, 1×10⁴ cells of BxPC-3 resulted in no metastasis in any of the six mice tested, while BxPC-3ML1 resulted in weak metastasis in 3 of the 5 mice tested (60% incidence). The injection of 1×10⁵ cells of BxPC-3 still resulted in no metastasis in the six mice tested in this experiment, while in case of BxPC-3LM1 all mice tested developed strong metastases.

The difference between the metastatic potential of BxPC-3 and BxPC-3LM1 is remarkable, since the latter was obtained by a single transplantation of the BxPC-3 cells in NOG mice.

### EXAMPLE 4

Gene Expression Analysis

As shown by the data set for in Table 5, BxPC-3 and BxPC-3LM1 were similar in their cell number doubling time, in the presence of micro-satellite markers (STR), and ras and p53 mutational status. Accordingly, in its key characteristics, BxPC-3LM1 showed no difference relative to the parental cell line.

The gene expression profiles of BxPC-3 and BxPC-3LM1 were then compared by microarray analysis (GeneChip Human Genome U133 Plus 2.0 Array, Affymetrix), essentially following the procedure described in Example 2.

Results

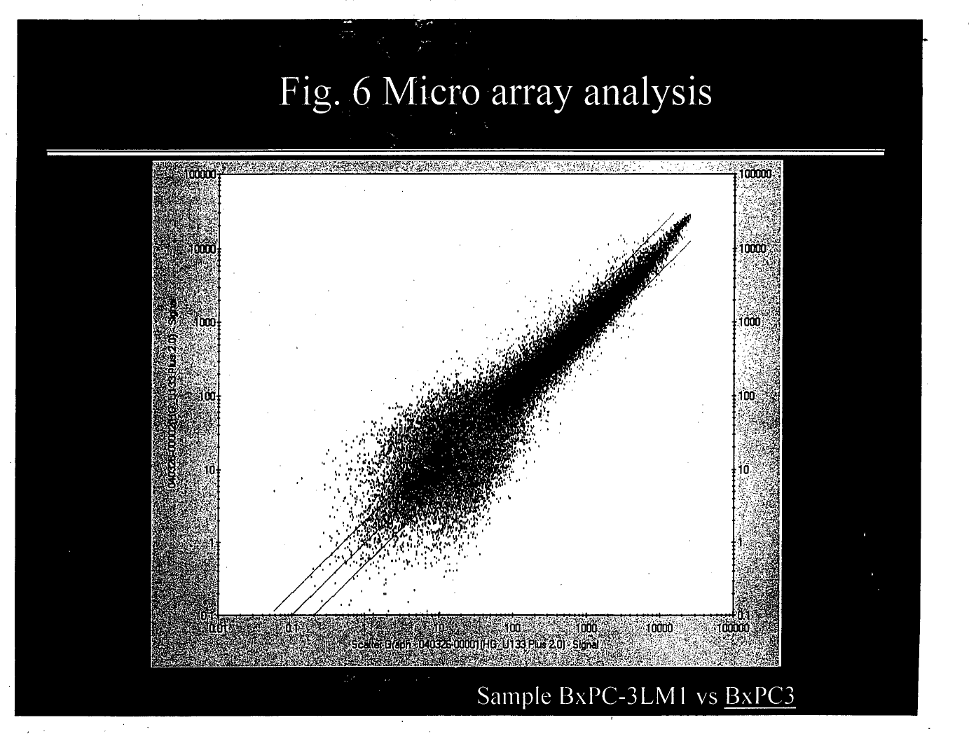

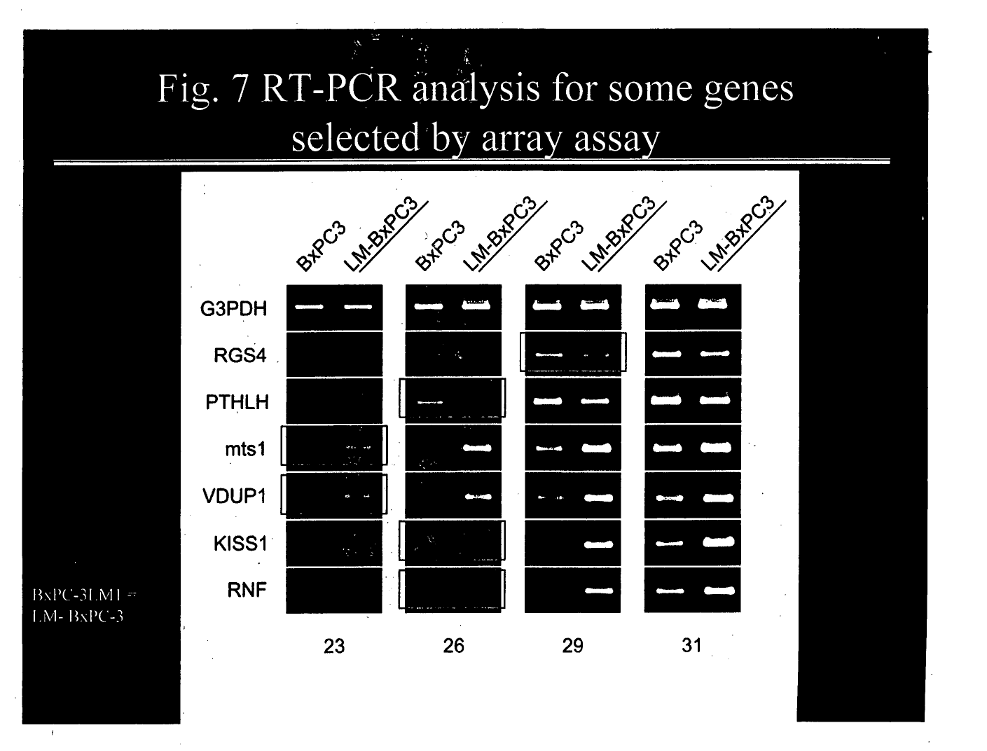

The results of the microarray analysis are shown in FIG. 6, where the signal intensity of each spot of the gene chip microarray is plotted. Table 6 lists all 84 genes selected with this microarray analysis. Some of the genes were further analyzed by RT-PCR. The results of this analysis are shown in FIG. 7.

Discussion

Based on a single transplantation experiment through transplanted in the NOD mice, 84 genes were identified with significantly different gene expressions in the original cell line BxPC-3 and the sub-line BxPC-3LM1 with high metastatic potential. While both cell lines were found to be originated from an identical cell line and to be equivalent pathologically and genetically, and in their in vitro growth. Therefore, the identification of significant differences of the gene expression patterns of the two cell lines are highly significant, and the differentially expressed genes are possibly responsible for metastasis. The differentially expressed genes identified are important targets for the development of drugs and therapeutic approaches for the prevention and treatment of tumor metastasis.

TABLE 1

| | | | | No. of mice with | |
|---|---|---|---|---|---|
| | | Cell dose | Autopsy | metastasis/total | Incidence |
| Cell line | Mice | (cells/head) | (week) | no. of mice (h) | (%) |
| MIA PaCa-2 | NOG | 1 × 10⁴ | 6 | 10/10 | 100.0 |
| pancreas; | | 1 × 10³ | 6 | 5/6 | 83.3 |
| adenocarcinoma | | 1 × 10² | 8 | 5/7 | 71.4 |
| | NOD/SCID | 1 × 10⁴ | 6 | 1/10 | 10.0 |
| | | 1 × 10³ | 6 | 0/7 | 0.0 |
| | | 1 × 10² | 8 | 0/6 | 0.0 |
| AsPC-1 | NOG | 1 × 10⁴ | 6 | 9/9 | 100.0 |
| pancreas; metastatic site: | | 1 × 10³ | 6 | 8/8 | 100.0 |
| ascites; adenocarcinoma | | 1 × 10² | 8 | 4/7 | 57.1 |
| | NOD/SCID | 1 × 10⁴ | 6 | 8/9 | 88.9 |
| | | 1 × 10³ | 6 | 1/8 | 12.5 |
| | | 1 × 10² | 8 | 0/6 | 0.0 |
| PANC-1 | NOG | 1 × 10⁴ | 6 | 8/8 | 100.0 |
| pancreas; | | 1 × 10³ | 6 | 6/8 | 75.0 |
| adenocarcinoma | | 1 × 10² | 8 | 3/8 | 37.5 |
| | NOD/SCID | 1 × 10⁴ | 6 | 0/10 | 0.0 |
| | | 1 × 10³ | 6 | 0/6 | 0.0 |
| | | 1 × 10² | 8 | 0/7 | 0.0 |
| Capan-1 | NOG | 1 × 10⁴ | 6 | 9/10 | 90.0 |
| pancreas; metastatic site: | | 1 × 10³ | 6 | 5/10 | 50.0 |
| liver; adenocarcinoma | | 1 × 10² | 8 | 0/8 | 0.0 |
| | NOD/SCID | 1 × 10⁴ | 6 | 0/10 | 0.0 |
| | | 1 × 10³ | 6 | 0/10 | 0.0 |
| | | 1 × 10² | 8 | 0/6 | 0.0 |
| BxPC-3 | NOG | 1 × 10⁵ | 6 | 8/8 | 100.0 |
| pancreas; | | 1 × 10⁴ | 6 | 1/8 | 12.5 |
| adenocarcinoma | NOD/SCID | 1 × 10⁵ | 6 | 0/8 | 0.0 |
| | | 1 × 10⁴ | 6 | 0/6 | 0.0 |
| Capan-2 | NOG | 1 × 10⁵ | 6 | 0/8 | 0.0 |
| pancreas; | | 1 × 10⁴ | 6 | 0/10 | 0.0 |
| adenocarcinoma | NOD/SCID | 1 × 10⁵ | 6 | 0/8 | 0.0 |
| PL45 | NOG | 1 × 10⁵ | 6 | 0/8 | 0.0 |
| Ductal adenocarcinoma; | | 1 × 10⁴ | 6 | 0/10 | 0.0 |
| pancreas | NOD/SCID | 1 × 10⁵ | 6 | 0/8 | 0.0 |

TABLE 2

| MRMCRC | | | | | | | | | |
|---|---|---|---|---|---|---|---|---|---|
| Miapaca, | | | | | | | | | |
| Panc1 >> | Gene Name | | | 2High | | | Low | 2H.AV - | Human |
| Capan2, PL45 | Miapaca, Panc1 >> Capan2, PL45 | Miapaca | Panc1 | AVG | Capan2 | PL45 | AVG | LAV | RNA |
| 4176 | TIS11B protein; butyrate response factor 1 (BRF1); | 5.351 | 5.311 | 5.331 | 0.000 | 0.000 | 0.000 | 5.331 | 0.000 |
| | EGF response factor 1 (E [symbol] | | | | | | | | |
| 6272 | prostate differentiation factor (PDF); macrophage | 3.389 | 3.375 | 3.382 | 0.000 | 0.000 | 0.000 | 3.382 | 0.000 |
| | inhibitory cytokine 1 (MIC1); | | | | | | | | |
| 6274 | glycoprotein hormone alpha subunit | 3.821 | 2.680 | 3.250 | 0.000 | 0.000 | 0.000 | 3.250 | 3.605 |
| 6153 | thrombopoietin (THPO); megakaryocyte colony | 3.579 | 2.921 | 3.250 | 0.000 | 0.000 | 0.000 | 3.250 | 3.265 |
| | stimulating factor; o-mpi ligand; | | | | | | | | |
| 6322 | manic fringe homolog (MFNG) | 3.578 | 2.757 | 3.167 | 0.000 | 0.000 | 0.000 | 3.167 | 3.021 |
| 6267 | complement component 5 (C5) | 3.911 | 2.373 | 3.142 | 0.000 | 0.000 | 0.000 | 3.142 | 2.893 |
| 5371 | jagged homolog 1 (JAG1; hJ1) | 3.913 | 2.356 | 3.135 | 0.000 | 0.000 | 0.000 | 3.135 | 1.980 |
| 4262 | interleukin enhancer-binding factor (ILF) ILF + | 3.209 | 3.029 | 3.119 | 0.000 | 0.000 | 0.000 | 3.119 | 0.000 |
| | interleukin enhancer binding factor | | | | | | | | |
| 4261 | PCAF-associated factor 65 alpha | 3.193 | 2.989 | 3.091 | 0.000 | 0.000 | 0.000 | 3.091 | 2.812 |
| 6227 | interleukin 12 alpha subunit (IL12-alpha; IL12A); | 3.376 | 2.779 | 3.076 | 0.000 | 0.000 | 0.000 | 3.076 | 0.000 |
| | cytotoxic lymphocyte maturati [symbol] | | | | | | | | |
| 4257 | nuclear respiratory factor 1 (NRF1); alpha | 2.893 | 3.178 | 3.035 | 0.000 | 0.000 | 0.000 | 3.035 | 2.606 |
| | palindromic binding protein | | | | | | | | |
| 6154 | stem cell factor (SCF); mast cell growth factor | 3.497 | 2.558 | 3.027 | 0.000 | 0.000 | 0.000 | 3.027 | 2.394 |
| | (MGF); o-kit ligand | | | | | | | | |
| 4251 | transcription repressor protein PRD1-8F1; beta- | 3.233 | 2.812 | 3.023 | 0.000 | 0.000 | 0.000 | 3.023 | 2.473 |
| | interferon gene positive regula [symbol] | | | | | | | | |
| 6144 | small inducible cytokine subfamily A member 1 | 3.341 | 2.691 | 3.016 | 0.000 | 0.000 | 0.000 | 3.016 | 0.000 |
| | (SCYA1); T-cell-secreted protei [symbol] | | | | | | | | |
| 2327 | Transducin beta-2 subunit; GTP-binding protein | 0.000 | 0.000 | 0.000 | 5.307 | 5.170 | 5.239 | −5.239 | 4.991 |
| | G(l)/G(s)/G(t) beta subunit 2 | | | | | | | | |
| 3366 | X-ray repair-complementing defective repair in | 0.000 | 0.000 | 0.000 | 4.023 | 3.457 | 3.740 | −3.740 | 4.468 |
| | Chinese hamster cells 1 (XRCC | | | | | | | | |
| 1433 | putative renal organic anion transporter 1 | 0.000 | 0.000 | 0.000 | 3.857 | 3.594 | 3.726 | −3.726 | 4.873 |
| | (hROAT1) | | | | | | | | |
| 1227 | G1/S-specific cyclin E (CCNE) | 0.000 | 0.000 | 0.000 | 3.840 | 3.435 | 3.637 | −3.637 | 4.686 |
| 4355 | retinoic acid receptor gamma (RARG) | 0.000 | 0.000 | 0.000 | 3.843 | 3.400 | 3.621 | −3.621 | 3.678 |
| 2437 | S100 calcium-binding protein A1; S-100 protein | 0.000 | 0.000 | 0.000 | 3.769 | 3.384 | 3.577 | −3.577 | 4.406 |
| | alpha chain | | | | | | | | |
| 1446 | neutral amino acid transporter A (SATT); alanine/ | 0.000 | 0.000 | 0.000 | 3.373 | 3.580 | 3.476 | −3.476 | 3.938 |
| | serine/cysteine/threonine tran [symbol] | | | | | | | | |
| 6434 | dopachrome tautomerase; dopachrome delta-isomerase | 0.000 | 0.000 | 0.000 | 3.536 | 3.332 | 3.434 | −3.434 | 4.262 |
| 4312 | ets transcription factor; NERF2 | 0.000 | 0.000 | 0.000 | 3.546 | 3.301 | 3.424 | −3.424 | 2.477 |
| 1443 | calcium-activated potassium channel beta subunit; | 0.000 | 0.000 | 0.000 | 3.680 | 2.944 | 3.312 | −3.312 | 4.554 |
| | maxl K channel beta subunit | | | | | | | | |
| 3232 | CD27BP (Siva) | 0.000 | 0.000 | 0.000 | 3.365 | 3.182 | 3.274 | −3.274 | 4.018 |
| 4442 | Keralin 10 (KRT10; K10) | 0.000 | 0.000 | 0.000 | 3.605 | 3.010 | 3.257 | −3.257 | 2.721 |
| 3374 | 6-O-methylguanine-DNA methyltransferase (MGMT); | 0.000 | 0.000 | 0.000 | 3.030 | 3.475 | 3.252 | −3.252 | 3.275 |
| | methylated-DNA-protein- | | | | | | | | |
| 3375 | xeroderma pigmentosum group A complementing | 0.000 | 0.000 | 0.000 | 4.284 | 2.200 | 3.242 | −3.242 | 3.592 |
| | protein (XPA) | | | | | | | | |
| 1347 | CDC6-related protein | 0.000 | 0.000 | 0.000 | 3.390 | 3.040 | 3.215 | −3.215 | 3.093 |
| 1312 | cell division protein kinase 4; cyclin-dependent | 0.000 | 0.000 | 0.000 | 3.201 | 3.205 | 3.203 | −3.203 | 3.332 |
| | kinase 4 (CDK4); PSK-J3 | | | | | | | | |
| 3432 | nociceptin receptor; orphanin FQ receptor; oploid | 0.000 | 0.000 | 0.000 | 3.432 | 2.967 | 3.200 | −3.200 | 4.165 |
| | receptor kappa 3 (OPRK3) | | | | | | | | |
| 5456 | cytochrome P450 XXVIIB1 (CYP27B1); 26-hydroxy- | 0.000 | 0.000 | 0.000 | 3.346 | 3.049 | 3.197 | −3.197 | 3.856 |
| | vitamin D-1-alpha-hydroxy [symbol] | | | | | | | | |
| 1174 | N-myc proto-oncogene | 0.000 | 0.000 | 0.000 | 3.323 | 3.012 | 3.167 | −3.167 | 3.466 |
| 1442 | solute carrier family 2 member 1 (SLC2A1); | 0.000 | 0.000 | 0.000 | 3.215 | 3.059 | 3.137 | −3.137 | 4.077 |
| | glucose transporter 1 (GLUT1) | | | | | | | | |
| 1324 | membrane-associated kinase myt1 | 0.000 | 0.000 | 0.000 | 3.859 | 2.379 | 3.124 | −3.124 | 3.645 |
| 3247 | casper, a FADD- and caspase-related inducer of | 0.000 | 0.000 | 0.000 | 3.631 | 2.659 | 3.095 | −3.095 | 4.255 |
| | apoptosis (CASH-alpha + CA | | | | | | | | |
| 1241 | C-src proto-oncogene (SRC1) | 0.000 | 0.000 | 0.000 | 3.316 | 2.829 | 3.072 | −3.072 | 2.795 |

TABLE 3
Metastatic potentials of BxPC-3 and
BxPC-3LM1 in liver of NOG mouse

| | | | No. of | Metastatic score | | | |
|---|---|---|---|---|---|---|---|
| Cell Dose | | Metastatic | mice | (head) | | | |
| (cells/head) | Cell line | incidence (%) | (head) | 0 | I | II | III |
| 1 × 10⁴ | BxPC-3 | 12.5 | 8 | 7 | 1 | 0 | 0 |
| | BxPC-3LM1 | 100.0 | 6 | 0 | 0 | 1 | 5 |
| 1 × 10⁵ | BxPC-3 | 100.0 | 8 | 0 | 8 | 0 | 0 |
| | BxPC-3LM1 | 100.0 | 6 | 0 | 0 | 0 | 6 |

TABLE 4
Metastatic potentials of BxPC-3 and
BxPC-3LM1 in liver of NOD/SCID mouse

| | | | No. of | Metastatic score | | | |
|---|---|---|---|---|---|---|---|
| Cell Dose | | Metastatic | mice | (head) | | | |
| (cells/head) | Cell line | incidence (%) | (head) | 0 | I | II | III |
| 1 × 10⁴ | BxPC-3 | 0 | 6 | 6 | 0 | 0 | 0 |
| | BxPC-3LM1 | 60 | 5 | 2 | 3 | 0 | 0 |
| 1 × 10⁵ | BxPC-3 | 0 | 6 | 6 | 0 | 0 | 0 |
| | BxPC-3LM1 | 100 | 6 | 0 | 0 | 1 | 5 |

TABLE 5
Checking of ras, p53 and other gene profiling by PCR

| | | BxPC3 (Parent) | LM-BxPC3 |
|---|---|---|---|
| | | Doubling time (hrs) | |
| | | 38.6 | 38.8 |
| STR markers | D5S818 | 143/143 | 143/143 |
| (size in bp) | D7S820 | 210/222 | 210/222 |
| | D13S317 | 182/182 | 182/182 |
| | D16S539 | 146/155 | 146/155 |
| ras gene | Ha-ras | CAT to CAC | CAT to CAC |
| mutation | | (codon 27) | (codon 27) |
| | K-ras | none | none |
| | N-ras | none | none |
| p53 gene | | TAT (Y) to TGT | TAT (Y) to TGT |
| mutation | | (C) (codon 20) | (C) (codon 20) |

BxPC-3LM1 = LM-BxPC-3

TABLE 6
Genes selected with array analysis

| | Signal | | | Signal | |
|---|---|---|---|---|---|
| Probe Set | Log | | Probe Set | Log | |
| Name | Ratio | description | Name | Ratio | description |
| 207526_s_at | −2.2 | interleukin 1 receptor-like 1 | 218963_s_at | 2.4 | DKFZP434G032 ( Keratin 23) |
| 211756_at | −2.3 | parathyroid hormone-like hormone | 202357_s_at | 3.5 | B-factor, properdin (complement) |
| 1556773_at | −2.3 | parathyroid hormone-like peptide | 219529_at | 2.4 | chloride intracellular channel 3 |
| 204337_at | −2.5 | regulator of G-protein signalling 4 | 206224_at | 2.0 | cystatin SN |
| 206300_s_at | −2.3 | parathyroid hormone-like hormone | 206199_at | 3.1 | carcinoembryonic antigen-related cell |
| | | | | | adhesionmolecule 7 |
| 231771_at | −2.1 | gap junction protein, beta 6 | 235515_at | 2.3 | |
| 204339_s_at | −2.3 | regulator of G-protein signalling 4 | 226622_at | 2.7 | KIAA 1359 (mucin 20) |
| 204338_s_at | −2.9 | regulator of G-protein signaling 4 | 220196_at | 3.4 | hypothetical protein (mucin 16 = CA125) |
| 206343_s_at | −2.3 | neuregulin 1 isoform SMDF | 208170_s_at | 2.3 | ring finger protein |
| 223779_at | −2.0 | Unknown | 205640_at | 2.0 | aldehyde dehydrogenase 3 family, |
| | | | | | member B1 |
| 211423_s_at | −2.1 | fungal sterol-C5-desaturase homolog | 209183_s_at | 2.7 | DKFZp564P1263 |
| 212977_at | −2.0 | G protein-coupled receptor | 208607_s_at | 2.4 | serum amyloid A2 |
| 222486_s_at | −2.2 | METH1 protein (ADAMTS1) | 213172_at | 2.0 | |
| 229004_at | −2.7 | METH1 protein (near ADAMTS15) | 205583_at | 2.4 | KiSS-1 metastasis-suppressor (KISS1), |
| | | | | | mRNA. (Nuclear) |
| 206237_s_at | −2.3 | neuregulin 1 isoform HRG-beta2 | 214458_x_at | 3.2 | serum amyloid A2-alpha |
| 235392_at | −2.2 | | 202161_at | 2.1 | protein kinase C-like 1 |
| 205535_s_at | −2.1 | BH-protocadherin (brain-heart) | 232105_at | 2.2 | |
| 201008_s_at | 2.4 | upregulated by 1,25-dihydroxyvitamin D-3 | 226960_at | 2.1 | |
| 212531_at | 2.4 | lipocalin 2 (oncogene 24p3) (LCN2) | 219508_at | 3.8 | glucosaminyl (N-acetyl) transferase 3, |
| | | | | | mucintype |
| 201010_s_at | 2.3 | upregulated by 1,25-dihydroxyvitamin D-3 | 1555349_a_at | 2.0 | integrin like protein, beta 2 (antigen CD18 |
| | | | | | (p95), lymphocyte |
| 201009_s_at | 2.7 | upregulated by 1,25-dihydroxyvitamin D-3 | 229659_s_at | 2.4 | |
| 207076_s_at | 2.8 | argininosuccinate synthetase (ASS) | 208125_s_at | 2.1 | kallikrein 8 |
| 209365_s_at | 2.2 | extracellular matrix protein 1 (ECM1) | 229927_at | 3.2 | EST (similar to lamina associated |
| | | | | | polypeptide 2, in nuclear |
| 203186_s_at | 3.3 | S100 calcium binding protein A4 (S100A4) | 35148_at | 2.0 | |
| 219795_at | 2.2 | solute carrier family 6 (neurotransmitter | 226158_at | 2.3 | |
| | | transporter), memb [symbol] | | | |
| 206421_s_at | 2.4 | serine (or cysteine) proteinase inhibitor, | 205597_at | 3.5 | NG22 protein |
| | | clade B (ovalbumin | | | |
| 201860_s_at | 2.5 | plasminogen activator, tissue (PLAT) | 1555203_s_at | 2.9 | NG22 (CTL4/NEU1 fusion protein) mRNA |
| | | | | | (sialidosis ) |
| 226071_at | 2.4 | Unknown (thrombospondin repeat | 219722_s_at | 2.3 | |
| | | containing 1 (TSRC1)) | | | |
| 225987_at | 2.4 | EST (DKFZp666M1410) | 231941_s_at | 2.8 | KIAA1359 (Mucin 20) |
| 202917_s_at | 2.9 | S100 calcium-binding protein A8 | 211906_s_at | 2.8 | SCCA2b (Squamous cell carcinoma antigen |
| | | (calgranulin A) (S100A8) | | | (SCCA) )SERF |
| 223179_at | 2.8 | EST (MGC: 10500) | 211848_s_at | 3.1 | carcinoembryonic antigen 2b |
| 219014_at | 2.0 | placenta-specific 8 (PLAC8) | 226560_at | 2.5 | sphingosine-1-phosphate phosphotase 2 |
| 202748_at | 2.1 | guanylate binding protein 2, | 227480_at | 2.5 | Susi domain containing2 |
| | | interferon-inducible | | | |
| 204259_at | 2.8 | matrix metalloproteinase 7 | 219630_at | 2.8 | epithelial protein up-regulated in carcinoma, |
| | | | | | membrane [symbol] |
| 213693_s_at | 2.1 | mucin 1, transmembrane | 204411_at | 2.3 | KIF21B kinesin family member 21B |
| 226755_at | 2.5 | EST (nasopharyngeal carcinoma-associated | 206198_s_at | 3.2 | carcinoembryonic antigen |
| | | antigen/LOC9 | | | |
| 204885_s_at | 2.0 | megakaryocyte potentiating factor precursor | 1553589_s_at | 2.1 | epithelial protein up-regulated in carcinoma, |
| | | | | | membrane [symbol] |
| 225283_at | 2.6 | arrestin domain containing 4 | 226147_s_at | 3.2 | polymeric immunoglobulin receptor/hepato- |
| | | | | | cellular carcinoma |
| 210029_at | 2.1 | interferon-gamma-inducible indoleamine | 205044_at | 2.9 | gamma-aminobutyric acid (GABA) A |
| | | 2,3-dioxygenase (I [symbol] | | | receptor, pi |
| 242649_x_at | 2.6 | | 205691_at | 3.3 | synaptogyrin 3 |
| 242907_at | 2.0 | | 220390_at | 4.9 | NM_024783.1 |
| | | | 202376_at | 5.2 | alpha-1-antichymotrypsin, precursor |
| | | | 205319_at | 4.0 | prostate stem cell antigen |

1. A method for testing tumor metastasis, comprising the steps of
(a) inoculating a tumor cell from a metastatic tumor or tumor cell line into a NOD/SCID/γ_{c}^{null} mouse, and
(b) monitoring the development of tumor metastasis.

2. The method of claim 1 wherein the tumor is carcinoma or sarcoma.

3. The method of claim 2 wherein the carcinoma is selected from the group consisting of pancreatic cancer, prostate cancer, breast cancer, colorectal cancer, gastrointestinal cancer, colon cancer, lung cancer, hepatocellular cancer, cervical cancer, ovarian cancer, liver cancer, bladder cancer, cancer of the urinary tract, thyroid cancer, renal cancer, carcinoma, melanoma, and brain cancer.

4. The method of claim 2 wherein the sarcoma is selected from the group consisting of liposarcomas, leiomyosarcomas, rhabdomyosarcoma, synovial sarcoma, angiosarcoma, fibrosarcoma, malignant peripheral nerve tumor, gastrointestinal stromal tumor, desmoid tumor, Ewing's sarcoma, osteosarcoma, chondrosarcoma, leukemia, lymphoma and myeloma.

5. The method of claim 1 wherein the metastasis is selected from the group consisting of hepatic, bone, brain, kidney, and lung metastases.

6. The method of claim 5 wherein the metastasis is hepatic metastasis.

7. The method of claim 6 wherein the tumor is selected from the group consisting of pancreatic cancer, breast cancer, colorectal cancer, and gastrointestinal cancer.

8. The method of claim 7 wherein the tumor cell is from a metastatic tumor cell line.

9. The method of claim 8 wherein the tumor cell line is a strongly metastatic tumor cell line.

10. The method of claim 8 wherein the cancer is pancreatic cancer, and the tumor cell line is selected from the group consisting of MIAPaCa-2, AsPC-1, PANC-1, Capan-1, and BxPC-3.

11. The method of claim 10 wherein the tumor cell line is selected from the group consisting of MIA_aCa-2, AsPC-1, and PANC-1.

12. The method of claim 1 wherein said tumor cell is inoculated into said mouse by portal vein injection.

13. The method of claim 12 wherein at least about 1×10² cells are inoculated.

14. The method of claim 12 wherein at least about 1×10³ cells are inoculated.

15. The method of claim 12 wherein at least about 1×10⁴ cells are inoculated.

16. The method of claim 1 wherein the development of tumor metastasis is monitored by observing the appearance and number of the metastatic nodules formed.

17. A method for testing a candidate compound for the treatment of tumor, comprising
(a) administering said candidate compound to a NOD/SCID/γ_{c}^{null} mouse which has developed tumor metastasis, and
(b) monitoring the effect of said candidate compound on said tumor metastasis.

18. The method of claim 17 wherein said metastasis is hepatic metastasis.

19. The method of claim 18 wherein said NOD/SCID/γ_{c}^{null} mouse has developed hepatic metastasis as a result of inoculation with a metastatic cancer cell line.

20. The method of claim 19 wherein said metastatic cancer cell line is selected from the group consisting of pancreatic, prostate, breast, colorectal, gastrointestinal, colon, lung, hepatocellular, cervical, ovarian, liver, bladder, urinary tract, thyroid, renal, carcinoma, melanoma, and brain cancer cell lines.

21. The method of claim 19 wherein the cancer cell line is a metastatic pancreatic adenocarcinoma cell line.

22. The method of claim 21 wherein said metastatic pancreatic adenocarcinoma cell line is selected from the group consisting of MIAPaCa-2, AsPC-1, PANC-1, Capan-1, and BxPC-3.

23. The method of claim 17 wherein said test compound is administered orally.

24. The method of claim 17 wherein said test compound is administered intravenously.

25. The method of claim 17 wherein said test compound is selected from the group consisting of peptides, polypeptides, antibodies and non-peptide small molecules.

26. A method comprising:
(a) introducing into a NOD/SCID/γ_{c}^{null} mouse a foreign gene, and
(b) monitoring the expression of said gene in said mouse.

27. The method of claim 26 wherein said foreign gene is introduced by a viral vector.

28. The method of claim 26 wherein said foreign gene is a gene differentially expressed in tumor metastasis.

29. The method of claim 28 wherein said tumor metastasis is hepatic metastasis.

30. The method of claim 29 wherein said hepatic metastasis is metastasis of pancreatic cancer.

31. The method of claim 30 wherein said foreign gene is selected from the group consisting of TIS1 1B protein; prostate differentiation factor (PDF); glycoproteins hormone α-subunit; thrombopoietin (THPO); manic fringe homology (MFNG); complement component 5 (C5); jagged homolog 1 (JAG1); interleukin enhancer-binding factor (ILF); PCAF-associated factor 65 alpha; interleukin-12 α-subunit (IL-12-α); nuclear respiratory factor 1 (NRF1); stem cell factor (SCF); transcription factor repressor protein (PRD1-BF1); and small inducible cytokine subfamily A member 1 (SCYA1).

32. The method of claim 30 wherein said foreign gene is selected from the group consisting of transducin β2 subunit; X-ray repair complementing defective repair in Chinese hamster cells 1; putative renal organic anion transporter 1; G1/S-specific cyclin E (CCNE); retinoic acid receptor-γ (RARG); S-100 calcium-binding protein A1; neutral amino acid transporter A (SATT); dopachrome tautomerase; ets transcription factor (NERF2); calcium-activated potassium channel β-subunit; CD27BP; keratin 10; 6-O-methylguanine-DNA-methyltransferase (MGMT); xeroderma pigmentosum group A complementing protein (XPA); CDC6-related protein; cell division protein kinase 4; nociceptin receptor; cytochrome P450 XXVIIB1; N-myc proto-oncogene; solute carrier family member 1 (SLC2A1); membrane-associated kinase myt1; casper, a FADD- and caspase-related inducer of apoptosis; and C-src proto-oncogene.

33. The method of claim 30 wherein said foreign gene is selected from the group consisting of interleukin 1 receptor-like 1, parathyroid hormone-like hormone, parathyroid hormone-like peptide, regulator of G-protein signaling 4, gap junction protein beta 6, neuregulin 1 isoform SMDF, fungal sterol-C5-desaturase homolog, G protein-coupled receptor, METH1 protein (ADAMTS1), METH1 protein (near ADAMTS15), BH-protocadherin (brain-heart), upregulated by 1,25-dihydroxyvitamin D-3, lipocalin 2 (oncogene 24p3) (LCN2), argininosuccinate synthetase (ASS), extrecellular matrix protein 1 (ECM 1), S100 calcium-binding protein A4 (S100Z4), solute carrier family 6 (neurotransmitter transporter) mem1, serine (or cysteine) proteinase inhibitor, clade B (ovalbumin), tissue plasminogen activator (PLAT), EST DKFZp666M1410, C100 calcium-binding protein A8 (calgranulin A) (S100A8), EST (MGC:10500), placenta-specific 8 (PLAC8), interferon-inducible guanylate binding protein 2, matrix metalloproteinase 7, mucin 1 (transmembrane), EST nasopharyngal carcinoma-associated antigen/LOC5, megakaryocyte potentiating factor precursor, arrestin domain containing 4, interferon-gamma-inducible indoleamine 2,3-dioxygenase, DKFZP434G031 (Keratin 23), B-factor (properdin, complement), chloride intracellular channel 3, cystatin SN, carcinoembryonic antigen-related cell adhesion molecule 7, KIAA1358 (mucin 20), hypothetical protein (mucin 16=CA125), ring finger protein, aldehyde dehydrogenase 3 family member B1, DKFZp564P1263, serum amyloid A2, KiSS-1 metastasis-suppressor (KISS1), serum amyloid A2-alpha, protein kinase C-like 1, glucosaminyl (N-acetyl) transferase 3, mucintype, integrin-like protein beta 2 (antigen CD18, p95), kallikrein 8, NG22 protein, KIAA1359 (mucin 20), SCA2b (squamous cell carcinoma antigen SCCA, SERPINB4), carcinoembryonic antigen 2b, sphingosine-1-phosphate phosphatase 2, Susi domain containing 2, epithelial protein up-regulated in carcinoma, KIF21B kinesin family member 21B, carcinoembryonic antigen, polymeric immunoglobulin receptor/hepatocelullar carcinoma, gamma-aminobutyric acid (GABA) A receptor p1, synaptogyrin 3, NM_{—}024 783.1, alpha-1-antichymotrypsin precursor, and prostate stem cell antigen.

34. The method of claim 28 further comprising the step of treating said mouse with a candidate anti-metastasis compound, and monitoring the expression level of said gene or its expression product as a result of said treatment.

35. The method of any one of claims 30-33 further comprising the step of treating said mouse with a candidate anti-hepatic metastasis compound, and monitoring the expression level of said gene or its expression product as a result of said treatment.

36. A method of establishing a tumor cell line with improved metastatic potential comprising
(a) introducing cells of a parental tumor cell line with low metastatic potential into a NOG mouse,
(b) allowing metastasis to develop, and
(c) isolating and propagating cells obtained from said metastasis in vitro to yield said cell line with improved metastatic potential.

37. The method of claim 36 further comprising the step of (d) introducing the cells propagated in step (c) into a NOG mouse; and (e) confirming the development of tumor metastasis.

38. The method of claim 36 wherein the metastasis is hepatic metastasis.

39. The method of claim 38 wherein the parental cell lines are of human origin.

40. The method of claim 39 wherein the cells are human pancreatic tumor cells.

41. The method of claim 40 wherein the metastasis is hepatic metastasis.

42. The method of claim 41 wherein the parental cell line is BxPC-3.

43. The method of claim 42 wherein the cell line with improved metastatic potential is BxPC-3LM1.

44. A cell line of human pancreatic cancer cells, having the metastatic and gene expression characteristics of BxPC-3LM1.

45. A method of screening a potential therapeutic agent for the prevention of treatment of metastatic pancreatic cancer, comprising administering said potential therapeutic agent to a cell line having the metastatic and gene expression characteristics of BxPC-3LM1, culturing the cells of said cell line, and determining whether said therapeutic agent inhibits the growth of said cells, proliferation of said cells or tendency of said cells to metastasize.

46. The method of claim 45 wherein said cell line is BxPC-3LM1.

47. A method of screening potential therapeutic agents for the treatment of metastatic pancreatic cancer in vivo comprising administering cells of a cell line having the metastatic and gene expression characteristics of BxPC-3LM1 to a mammalian host, allowing said cells to proliferate in said host, and administering said therapeutic agent to said host, and examining said host to determine whether said therapeutic agent inhibits the growth, proliferation or metastasizing of said pancreatic cancer cells.

48. The method of claim 47 wherein said cell line is BxPC-3LM1.

49. The method of claim 47 wherein the mammalian host is a mouse.

50. The method of claim 49 wherein the mouse is a NOG mouse.
